(19) United States
(12) Patent Application Publication
(10) Pub. No.: US 2018/0150604 A1
(43) Pub. Date: May 31, 2018

(54) Method and System for Determining The Nursing Workload of Care Workers

(71) Applicant: Istituto Mediterraneo per i Trapianti e Terapie ad Alta Specializzazione S.r.l., Palermo (IT)

(72) Inventors: Giuseppe Arena, Palermo (IT); Michelangelo Calo, Palermo (IT); Maria Angela Barone, Palermo (IT); Andrea Lepre, Palermo (IT); Santa Giammona, Palermo (IT); Maria Rosaria Tarantino, Palermo (IT)

(21) Appl. No.: 15/362,237

(22) Filed: Nov. 28, 2016

(51) Int. Cl. G06F 19/00; G06Q 10/06

(52) U.S. Cl. CPC G06F 19/327; G06Q 10/063118; G06F 19/322; G06Q 10/06393

(57) ABSTRACT

A method and system for determining the nursing workload of care workers is provided for receiving health care requirement data of at least one patient, selecting at least one nursing work task with high nursing care related to the health care requirement data, and assigning a nursing care score to the at least one selected nursing work task, so as to level out the nursing workload among care workers and to optimise the assignment of patients to each care worker.

### BACKGROUND OF THE INVENTION

### Field of the Invention

The field of the invention relates to a method and system for determining the nursing workload of care workers. More specifically, the present invention relates to a method and system for determining the nursing workload of care workers by assigning a nursing care score to the at least one selected nursing work task, so as to level out the nursing workload among care workers and to optimise the assignment of patients to each care worker.

### Brief Description of the Related Art

Resourcing of nursing care is a concern for nursing managers across the globe today and therefore understanding the level of the nursing workload enables appropriate resource planning.

The term “nursing workload” relates to the amount of work or performance required to carry out nursing activities in a specified time on a patient. Nursing workload quantifies nursing work to manage and allocate nursing staff in a unit/department and includes the totality of work activities carried out by the nursing staff in the unit/department.

A known workload measuring system is described in the U.S. Pat. No. 8,392,232 B2, which provides real time assessment and management of patient acuity information to identify required care staff (and associated skills) by using data collected from multiple patients to assign a weighted value to a data element that accurately reflects the importance and complexity of each data source in measuring patient acuity. The system predicts healthcare worker workload using an acquisition processor to acquire multiple data items associated with care requirements of a particular patient from multiple different sources. The known system comprises a data processor determines an acuity score of the particular patient by determining a single score comprising a combination of weighted individual score values derived from corresponding individual items of the multiple data items. The known system further comprises a translation processor interprets determined acuity score to provide an estimated healthcare worker workload for meeting the care requirements of the patient by using predetermined translation data associating acuity score with corresponding healthcare worker workload.

There are further known workload measuring systems for measuring the nursing workload. These systems include the Therapeutic Interventions Scoring System (TISS). The Therapeutic Interventions Scoring System-28 (TISS-28) is a tool used to assign scores to patients according to severity of illness of the patient. In TISS-28, the number of therapeutic interventions, and the amount of nursing time spent on the patient, is related to the severity of the patient's clinical condition. The TISS-28 score is, therefore, an indicator of the nursing workload required in a setting of an intensive care unit (ICU). The Nursing Activities Score (NAS) has been developed as a result of modifications to the TISS-28 with an additional five items, plus 14 sub-items. The NAS has a reduced scale of 23 items in total. The NAS scale assesses the nursing activities performed on and care provided to critically ill patients by the nursing staff. Each item in the NAS has an assigned score, and the sum of all the scores provides a total that represents the percentage of time that the nurse spends in direct care of each patient over a 24-hour period in the ICU.

The nursing staff is expected to use the workload measurement systems to collect data related to the nursing work they perform. These prior art systems often add to nurses' workload by requiring the nurse to describe the nursing work performed. This method is inefficient and is often perceived by the nurses as an extra task and not taken as seriously as it should be taken. For example, the NAS has its own specific instructions for use, and the use of this tool adds to the burden of providing nursing care, requires additional activity beyond documenting patient care, and requires the nurses to understand both the NAS methodology and the software system. Further, the separating of “monitored versus non-monitored patients”, where the nurse/patient ratio for non-monitored patients is 1:5 and the nurse/patient ratio for monitored patients is 1:3, is also not reliable enough to identify the level of complexity of nursing care required by the patients and does not consider nursing workload and intensity measurement. Hence, the prior art methods do not match the current level of patient care complexity and the workload involved with an assigned patient. This situation can result in nurse dissatisfaction, with negative effects on quality of the nursing care and patient satisfaction. Hence, an appropriate system of measuring nursing resource intensity and workload would be a desired system that measures elements of nurses' work that influence nursing workload and patient outcomes.

This disclosure teaches a method and system for determining the nursing workload of care workers, such as nurses, which can be integrated into the existing patient documentation system (e.g., actual electronic health record (EHR)) without the need for entering data on a separate workload system and obviate additional nursing tasks. The nurses' tasks do not increase and the nurses are only required to pay more attention to documenting patients' needs and clinical conditions. The workload distribution may lead to a reduction in stress and in burnout syndrome among nurses, and to improvements in job satisfaction, and consequently better retention in the workplace. Furthermore, an improved proportion of registered nurses is associated with positive outcomes, such as lower rates of medication errors and hospital-acquired infections

### SUMMARY OF THE INVENTION

In view of the state of the known technology, the forgoing needs are met, to a great extent, by the present invention. In accordance with one aspect a method for determining the nursing workload of care workers is provided that comprises the steps of receiving health care requirement data of at least one patient, selecting at least one nursing work task with high nursing care related to the health care requirement data, and assigning a nursing care score to the at least one selected nursing work task.

In another aspect, the method further comprises calculating a total nursing care score from one or more of the assigned nursing care scores.

In another aspect, the method further comprises dividing the total nursing care score by the number of patients of at least one group of care workers in order to obtain an average nursing care score of the at least one group of care workers.

In another aspect, the method further comprises assigning the care workers to a group of patients, such that each care worker has the same average nursing care score, a number of patients grouped according to the specific characteristics of a relevant health care unit/department, a workload that is within a predetermined maximum nursing care score, the same group of patients for each period of nursing shift, in order to grant continuity of empathy and care between care worker and patient, patients who needs a nursing care workload consistent with the maximum nursing care score assigned to each care worker according to the work capacity identified by the occupational physician, not more than one new admitted patient, at least patients who are hospitalised in neighbouring rooms, at least patients who have similar isolation condition, patients who have isolation condition consistent with the health condition of the care workers.

In another aspect, the assigned nursing work task of the method has a value from 1.5 to 5.0.

In another aspect, the value of the assigned nursing work task of the method is adjusted in the case a patient is isolated.

In another aspect, the method further comprises notifying a user (for example the care coordinator) of at least one of the average nursing care score of the group of care workers and/or of the nursing care score of each individual patient and/or of the average nursing care score for each health care unit/department.

In another aspect, the assigned nursing work tasks of the method are further categorized according to the skill level, the work capacity, and the health and physical condition of the care workers.

In another aspect, the selected ones of the assigned nursing work tasks are assigned based on at least one of the presence of care workers or the skill level of the present care workers within the hospital, the same average nursing care score for each care workers, the specific characteristic of the relevant health care unit/department, a workload that is within a predetermined maximum nursing care score, patients who needs a nursing care workload consistent with the maximum nursing care score assigned to a specific care worker, which is determined according to the work capacity identified by the occupational physician, the allocation of not more than one new admitted patient for each care workers, the allocation of the same group of patients for each period of nursing shift, in order to grant continuity of empathy and care between care worker and patient, at least patients who are hospitalised in neighbouring rooms, at least patients who have similar isolation condition, patients who have isolation condition consistent with the health condition of the care workers.

In another aspect, the method comprises receiving of the health care requirement data, which are a selection of observation documented in the “RN Assessment and PoC” (Registered Nurse Assessment and Plan of Care) clinical document once the care worker has performed his/her assessment of the patient, through the data extraction of patient health record.

In another aspect, the method comprises assigning the care workers to a group of patients such that a human resource management system identifies them as at work within the hospital regardless of the unit/department where a care worker usually performs his/her job tasks, in order to level out the nursing workload among all the care workers within the hospital.

In accordance with another aspect of the present invention, a system for determining the nursing workload of care workers is provided that comprises a patient database having a plurality of health care requirement data of at least one patient, a nursing work task database having a plurality of nursing activities with high nursing care, an acquisition processor for selecting at least one nursing work task with high nursing care of the nursing work task database related to the health care requirement data, an acquisition processor for assigning a nursing care score to the at least one selected nursing work task with high nursing care, a database having the list of the available care workers, an acquisition processor to identify the care workers within the hospital in each nursing shift and theirs most accurate work capacity, skill, health and physical conditions, a data processor for calculating a total nursing care score from one or more of the assigned nursing care scores, a data processor to assign each care worker a specific group of patients, and a shift report generator that sends automatically to the user (for example the care coordinator)—before the beginning of the nursing shift—a report that assigns to each care worker a specific group of patients.

In another aspect, the system is configured such that the data processor is adapted to calculate an average nursing care score of at least one group of care workers by dividing the total nursing care score by the number of patients.

In another aspect, the system is configured such that the acquisition processor is adapted to assign one or more of the care workers to a group of patients such that the care workers have substantially the same average nursing care score, a number of patients based upon the specific characteristic of the relevant health care unit/department, a workload that is within a predetermined maximum nursing care score, the same group of patients for each period of nursing shift, in order to grant continuity of empathy and care between care worker and patient, patients who needs a nursing care workload consistent with the maximum nursing care score assigned to a specific care worker, which is determined according to the work capacity identified by the occupational physician, not more than one new admitted patient, patients who are hospitalised in neighbouring rooms, patients who have similar isolation condition, patients who have isolation condition consistent with the health condition of the care workers.

In another aspect, the system is configured such that the nursing care score of the assigned nursing work tasks has a value from 1.5 to 5.0.

In another aspect, the system is configured such that the data processor is adapted to monitor the calculated total nursing care score to identify critical situations. A critical situation occurs when the total nursing care score for each care worker exceeds the expected maximum nursing care score for each care worker. Accordingly, the alert informs the user (for example the care coordinator) that a new care worker is required in order to reduce the actual nursing workload of care for each care worker to the maximum nursing workload of care that can be assigned to each care worker.

In another aspect, the system is configured such that the data processor is adapted to interpret the assigned nursing work tasks for at least one patient to identify the required skill level, health and work capacity of the care workers and compare the required skill level, health and work capacity of the care workers with an existing available skill level, health and work capacity of the care workers.

In another aspect, the system further comprises an alert generator for generating an alert message to a user in response to any identified critical situation.

In accordance with yet another aspect of the present invention, a computer program product stored on a non-transitory computer readable medium is provided, comprising a first logic for receiving health care requirement data of at least one patient, a second logic for selecting at least one nursing work task with high nursing care related to the health care requirement data, a third logic for assigning a nursing care score to the at least one selected nursing work task with high nursing care, a fourth logic for calculating a total nursing care score from one or more of the assigned nursing care scores, a fifth logic for receiving care workers data, and a sixth logic for assigning patients to care workers according to above system.

In another aspect, the computer program product is configured such that first logic is capable of adjusting the nursing care score for work tasks in the case a patient is isolated.

### BRIEF DESCRIPTION OF THE DRAWINGS

The invention will now be described on the basis of figures. It will be understood that the embodiments and aspects of the invention described in the figures are only examples and do not limit the protective scope of the claims in any way. The invention is defined by the claims and their equivalents. It will be understood that features of one aspect or embodiment of the invention can be combined with a feature of a different aspect or aspects of other embodiments of the invention. This invention becomes more obvious when reading the following detailed descriptions of some examples as part of the disclosure under consideration of the enclosed drawings. Referring now to the attached drawings which form a part of this disclosure:

FIG. 1 is an exemplary collaboration diagram of a communication network for determining the nursing workload of care workers according to one embodiment of the present invention.

FIG. 2 is an exemplary nursing care score report workflow of one embodiment of the present invention.

FIG. 3 is a block diagram illustrating a method for determining the nursing workload of care workers according to one embodiment of the present invention.

### DETAILED DESCRIPTION

The object of the present invention is fully described below using examples for the purpose of disclosure, without limiting the disclosure to the examples. The examples present different aspects of the present invention. To implement the present technical teaching, it is not required to implement all of these aspects combined. Rather, a person skilled in the art will select and combine those aspects that appear sensible and required for the corresponding application and implementation.

As can be seen in the table 1 below, a total of 53 nursing work tasks with high patient care complexity were identified. Nursing work tasks requiring only minimal nursing care were excluded from this table and are not included in the system and method at this stage. It would be possible to include these additional nursing work tasks if required at a later stage.

The Nursing Care Score (NCS) is based on a hierarchical classification of nursing work tasks/conditions and is set out in the table below.

TABLE 1
Nursing Work Tasks/Conditions with Mean Relevance Scores and
Assigned Nursing Care Scores (NCS)
NCS Observation Mapping Table

| Item Number | Nursing Work Tasks/Conditions | NCS |
|---|---|---|
| 1 | Uncontrolled agitation | 5.0 |
| 2 | Disoriented to person, place, and time | 5.0 |
| 3 | Restraints | 5.0 |
| 4 | Totally passive mobilization with assistance | 5.0 |
| | devices | |
| 5 | Continuous hemofiltration | 4.5 |
| 6 | Noncompliant | 4.5 |
| 7 | Tracheostomy | 4.5 |
| 8 | Complex dressing | 4.0 |
| 9 | Obtunded | 4.0 |
| 10 | Insulin drip | 4.0 |
| 11 | Transvenous pacemaker | 4.0 |
| 12 | Non-invasive mechanical ventilation | 4.0 |
| 13 | Incontinent (gastrointestinal) | 4.0 |
| 14 | Incontinent (genitourinary) | 4.0 |
| 15 | Totally dependent for personal hygiene | 4.0 |
| 16 | Mobilization with average aid | 3.5 |
| 17 | Anxious | 3.5 |
| 18 | Limited attention span | 3.5 |
| 19 | Responds inappropriately | 3.5 |
| 20 | Pain | 3.5 |
| 21 | Febrile | 3.5 |
| 22 | Vital signs every 2 hours | 3.5 |
| 23 | Heparin drip | 3.5 |
| 24 | Ventricular assist device | 3.5 |
| 25 | Flexiseal | 3.5 |
| 26 | Bladder irrigation | 3.5 |
| 27 | Intake and output < every 4 hours | 3.0 |
| 28 | Nitroglycerin drip | 3.0 |
| 29 | Blood sample, more than once every shift | 3.0 |
| 30 | External/transcutaneous pacemaker | 3.0 |
| 31 | Chest tube | 3.0 |
| 32 | Faecal bag | 3.0 |
| 33 | Depressed | 2.5 |
| 34 | Arterial line | 2.5 |
| 35 | Mediastinal drainage | 2.5 |
| 36 | Enteral nutrition | 2.5 |
| 37 | Vital signs every 4 hours | 2.5 |
| 38 | Pigtail catheter | 2.5 |
| 39 | Parenteral nutrition | 2.5 |
| 40 | Nasogastric tube | 2.5 |
| 41 | Percutaneous Endoscopic Gastrostomy tube | 2.5 |
| 42 | Feeding tube | 2.5 |
| 43 | “T” tube | 2.5 |
| 44 | Medium dressing | 2.5 |
| 45 | Partially dependent for personal hygiene | 2.5 |
| 46 | Central venous catheter | 2.0 |
| 47 | Blood sampling every shift (ABG, VBG) | 2.0 |
| 48 | Mobilization with minimal help | 2.0 |
| 49 | Jackson Pratt n°1 | 2.0 |
| 50 | Jackson Pratt n°2 | 2.0 |
| 51 | Jackson Pratt n°3 | 2.0 |
| 52 | Simple dressing | 2.0 |
| 53 | Vital signs every 8 hours | 1.5 |

Each one of the nursing work tasks in Table 1 is assigned to a final NCS. The NCS consists of 53 items that assess the activities performed and care provided to patients by the individual nurses (i.e., care workers). Each one of the items has an assigned value from 1.5 to 5.0, and the total score is the sum of all the values. The sum of values, related to the 53 items of the scale, will have a value in the range from “0” to “170”. The sum of all the patients' scores in a unit/department of a hospital is then divided by the number of patients staying in the unit/department, so as to obtain the average NCS of the relevant specific unit/department. The average NCS is used for nursing patient assignments in such a way that each nurse has an NCS as close as possible to the average NCS.

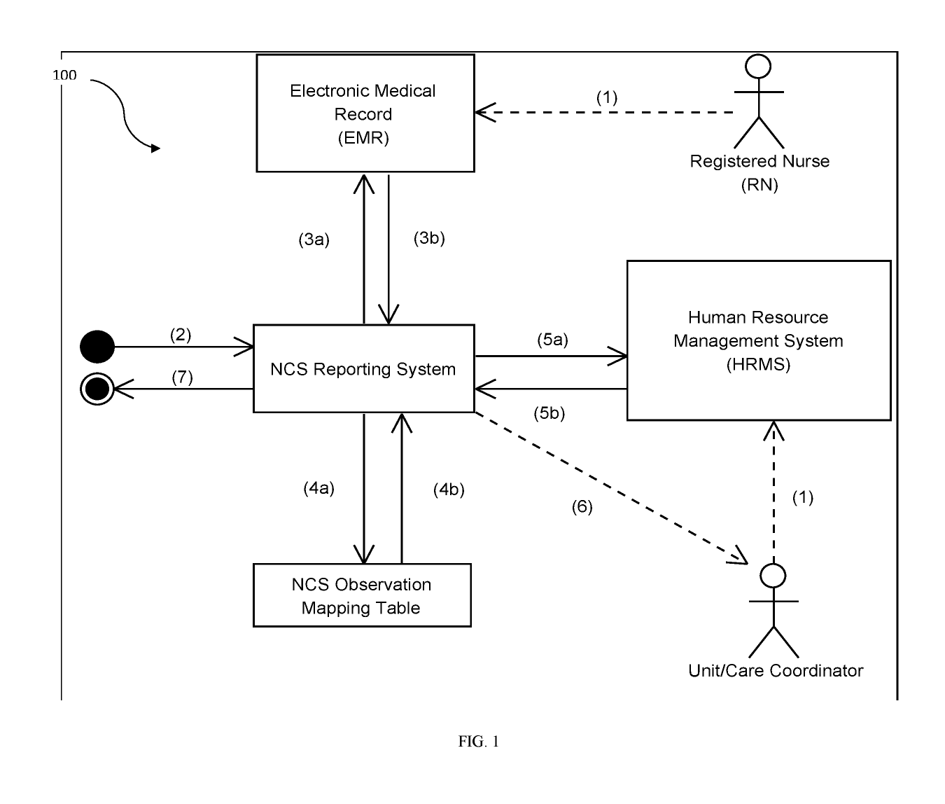

As can be seen in FIG. 1, a communication network is shown for generating a nursing workload report. A nursing assessment of all registered nurses (RNs) or other care workers is done at each the turnover of the nursing staff. The nurses take an assessment of the patients present on the unit/department during each shift. The nurses fill out the “RN Assessment and PoC” clinical document, which are stored in the Electronic Medical Record (EMR), for each one of the patients on the unit/department (Step No. 1). Each month a user (for example the care coordinator) enters the nursing shift into a human resource management system (HRMS) and updates the HRMS whenever there are changes (Step No. 1). One hour before the beginning of each nursing shift the NCS Reporting System becomes active (Step No. 2), collects the health care requirement data from the EMR (Steps No(s) 3a and 3b), identifies the nursing work task by virtue of the NCS Observation Mapping Table (Steps No(s) 4a and 4b), and computes the NCS for each patient. Afterwards the NCS Reporting System collects the list of care workers, who will be present during the subsequent nursing shift, from the Human Resource Management System (HRMS) (Steps No(s) 5a and 5b) and will automatically assign the patients to the care workers. Finally, a nursing workload report is generated and sent to a user (for example the care coordinator) (Step No. 6) and hence the process ends until the beginning of the next shift, when the described process will start again (Step. No. 7).

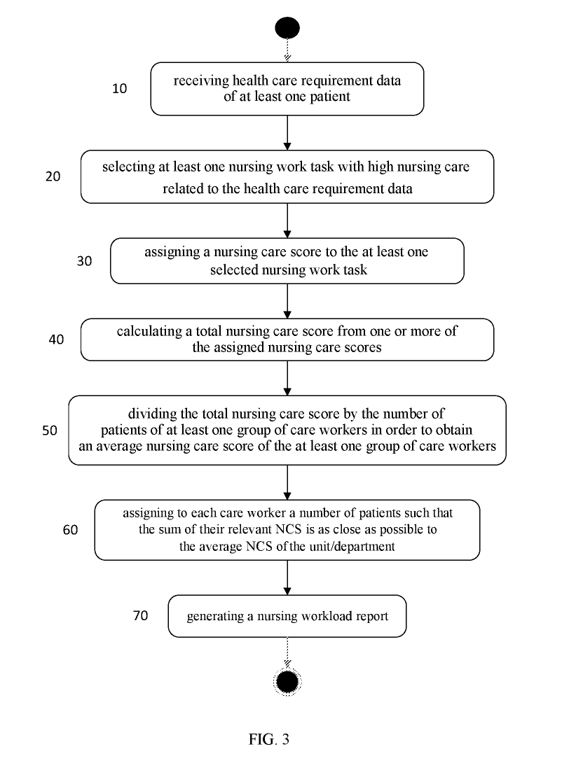

It can be seen in FIG. 3 that health care requirement data of at least one of the patients is received by an electronic medical record system, as shown in method step 10. At least one nursing work task with high nursing care related to the health care requirement data is selected for each patient, method step 20. By virtue of a mapping table (NCS Observation Mapping Table), which allows to link to each health care requirement a specific nursing work task, the nursing care score (NCS) of the at least one selected nursing work task is assigned to each patient, method step 30. The method further comprises the step of calculating a total nursing care score for the unit/department from one or more of the assigned nursing care scores, method step 40. The method comprises the method step 50 of dividing the total nursing care score by the number of patients of at least one group of care workers in the unit/department, in order to obtain an average nursing care score of the at least one group of care workers. The method comprises the method step 60 of assigning to each care worker a number of patients such that the sum of their relevant NCS is as close as possible to the average NCS of the unit/department, taking also into account the constraints described in point 38 below. The method comprises the method step 70 of generating a nursing workload report.

All of the registered nurses or the other care workers in each unit/department are assigned to a group of patients in one of the following criteria, each of the care workers has at least one among the group consisting of the same average nursing care score, a number of patients grouped according to the specific characteristics of a relevant health care unit/department, a workload that is within a predetermined maximum nursing care score, the same group of patients for each period of nursing shift, in order to grant continuity of empathy and care between care worker and patient, patients who needs a nursing care workload consistent with the maximum nursing care score assigned to a specific care worker, which is determined according to the work capacity identified by the occupational physician, not more than one new admitted patient, at least patients who are hospitalised in close rooms, at least patients who have similar isolation condition, and patients who have isolation condition consistent with the health condition of the care workers.

It was noted above with reference to the table 1 that the nursing care score of the assigned nursing work tasks has a value from 1.5 to 5.0. The selected nursing work tasks are categorized according to the skill level, the work capacity, and the health and physical condition of each nurse (i.e. care worker) in the unit/department. The receiving of the health care requirement data is implemented by data extraction of the patient health record, wherein the nurses (i.e. care workers) are assigned to a group of patients such that a human resource management system identifies them as at work within the hospital regardless of a unit/department where a care worker usually performs his/her job tasks, in order to homogenise the nursing workload among all the care workers within the hospital.

As can be seen in FIG. 1, the system 100 comprises a patient database 1 (EMR) having a plurality of health care requirement data of at least one patient. The system 100 further comprises a nursing work task database 2 (NCS Observational Mapping Table) having data on a plurality of nursing activities with a high nursing care (NCS). The system 100 further comprises an acquisition processor 3 for selecting at least one nursing work task with high nursing care of the nursing work task database 2 related to the health care requirement data and for assigning a nursing care score (NCS) to the at least one selected nursing work task with high nursing care (NCS).

The acquisition processor 4 identifies the nurses or other care workers within the hospital in each nursing shift and their work capacity, skill, health and physical conditions.

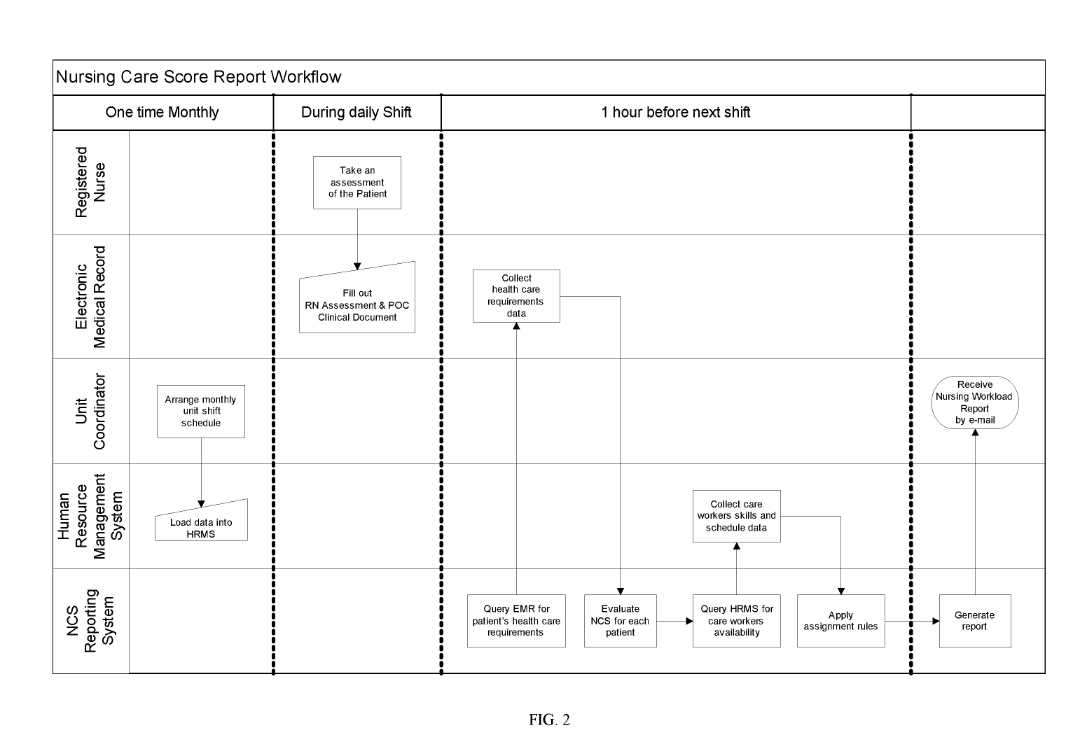

The system 100 further comprises a data processor 5 for calculating a total nursing care score from one or more of the assigned nursing care scores, as outlined in method step 50 of FIG. 2. Further, the data processor 5 assigns one or more of the care workers to a specific group of patients, for example in a nursing unit/department. A shift report generator 6 sends automatically to a care coordinator—before the beginning of the nursing shift—a report that assigns the care workers in the unit/department to a specific group of patients.

The data processor 5 of the present invention is adapted to monitor the calculated total nursing care score to identify critical situations. The data processor 5 is further adapted to evaluate with respect to each nurse the calculated total nursing care score in order to identify the required skill level, health and work capacity of the care workers and compare the required skill level, health, and work capacity of the care workers with the available skill level, health, and work capacity of the care workers.

The system 100 further comprises an alert generator 7 for generating an alert message to a user in response to any one of the identified critical situation, as explained above.

Again, as can be seen in FIG. 2, at the moment of the hiring of a nurse and whenever a nurse earns a new qualification, the human resources office updates the nurse's data stored in the HRMS. In addition, each month the care coordinator enters the nursing shift into the HRMS and update it whenever there are changes. All registered nurses take an assessment during the daily shift of the patients present on the unit/department, fill in the different field of observation in the EMR and in the “RN Assessment and PoC” clinical document. One hour before the beginning of each nursing shift the NCS Reporting System becomes active and enquiries the EMR in order to extract the health care requirement date for all patients within each unit/department. These data are analysed and, on the basis of the nursing work task database 2 (NCS Observational Mapping Table), the nursing work task of each patient and the relevant nursing care score are selected. Simultaneously the overall NCS of each patient and the average NCS of each unit/department are calculated. Subsequently, the NCS Reporting System enquiries the HRMS in order to extract data of the nurses, who will be present in the next shift, and their skills. Afterwards the NCS Reporting System assigns each nurse the group of patients, on the basis of the average NCS of the relevant unit/department and taking into account the presence of any constraints (for example incompatibility between nursing work task and skills and/or limitations of a nurse). Finally, a nursing workload report is generated and sent to a user (for example a care coordinator).

From the above description of the present invention, those skilled in the art will perceive improvements, changes, and modifications on the present invention. Such improvements, changes, and modifications within the skill in the art are intended to be covered by the appended claims.

1. A method for determining the nursing workload of care workers, comprising the steps of:
receiving health care requirement data of at least one patient;
selecting at least one nursing work task with high nursing care related to the health care requirement data; and
assigning a nursing care score to the at least one selected nursing work task.

2. The method according to claim 1, further comprising the step of calculating a total nursing care score from one or more of the assigned nursing care scores.

3. The method according to claim 1, further comprising the step of dividing the total nursing care score by the number of patients of at least one group of care workers in order to obtain an average nursing care score of the at least one group of care workers.

4. The method according to claim 3, further comprising the step of assigning the care workers to a group of patients such that each care worker has
a. the same average nursing care score;
b. a number of patients grouped according to the specific characteristics of a relevant health care unit/department;
c. a workload that is within a predetermined maximum nursing care score;
d. the same group of patients for each period of nursing shift, in order to grant continuity of empathy and care between care worker and patient;
e. patients who needs a nursing care workload consistent with the maximum nursing care score assigned to a specific care worker, which is determined according to the work capacity identified by the occupational physician;
f. not more than one new admitted patient;
g. at least patients who are hospitalised in close rooms;
h. at least patients who have similar isolation condition; and
i. patients who have isolation condition consistent with the health condition of the care workers.

5. The method according to claim 1, wherein the nursing care score of the assigned nursing work tasks has a value from 1.5 to 5.0.

6. The method according to claim 1, wherein the value of the assigned nursing work task is adjusted in the case a patient is isolated.

7. The method according to claim 4, further comprising the step of notifying a user of at least one of the average nursing care score of the group of care workers and/or of the nursing care score of each individual patient, and/or of the average nursing care score for each health care unit/department.

8. The method according to claim 1, wherein the selected nursing work tasks are categorized according to the skill level, the work capacity, and the health and physical condition of the care workers.

9. The method according to claim 1, wherein selected nursing work tasks are assigned based on
a. at least one of the presence of care workers or the skill level of the present care workers within the hospital;
b. the same average nursing care score for each care workers;
c. the specific characteristic of the relevant health care unit/department;
d. a workload that is within a predetermined maximum nursing care score;
e. patients who needs a nursing care workload consistent with the maximum nursing care score assigned to a specific care worker, which is determined according to the work capacity identified by the occupational physician;
f. the allocation of not more than one new admitted patient for each care workers;
g. the allocation of the same group of patients for each period of nursing shift, in order to grant continuity of empathy and care between care worker and patient;
h. at least patients who are hospitalised in neighbouring rooms;
i. at least patients who have similar isolation condition; and
j. patients who have isolation condition consistent with the health condition of the care workers.

10. The method according to claim 1, further comprising the method step of receiving of the health care requirement data, which are a selection of observation documented in the RN Assessment and PoC (Registered Nurse Assessment and Plan of Care) once the care worker has performed his/her assessment of the patient, through the data extraction of a patient health record.

11. The method according to claim 1, further comprising the step of assigning the care workers to a group of patients such that a human resource management system identifies the care workers as at work within the hospital regardless of the unit/department where a care worker usually performs his/her job tasks, in order to level out the nursing workload among all the care workers within the hospital.

12. A system for determining the nursing workload of care workers, comprising:
a patient database having a plurality of health care requirement data of at least one patient;
a nursing work task database having a plurality of nursing activities with high nursing care;
an acquisition processor for selecting at least one nursing work task with high nursing care of the nursing work task database related to the health care requirement data;
an acquisition processor for assigning a nursing care score to the at least one selected nursing work task with high nursing care;
a database having the list of the available care workers;
an acquisition processor to identify the care workers within the hospital in each nursing shift and theirs most accurate work capacity, skill, health and physical conditions;
a data processor for calculating a total nursing care score from one or more of the assigned nursing care scores;
a data processor to assign each care worker a specific group of patients; and
a shift report generator that sends automatically to the user (for example the care coordinator—before the beginning of the nursing shift—a report that assigns to each care worker a specific group of patients.

13. The system according to claim 11, wherein the data processor is adapted to calculate an average nursing care score of at least one group of care workers by dividing the total nursing care score by the number of patients.

14. The system according to claim 11, wherein the acquisition processor is adapted to assign one or more of the care workers to a group of patients such that the care workers have at least one of
a. the same average nursing care score;
b. a number of patients based upon the specific characteristic of the relevant health care unit/department;
c. a workload that is within a predetermined maximum nursing care score;
d. the same group of patients for each period of nursing shift, in order to grant continuity of empathy and care between care worker and patient;
e. patients who needs a nursing care workload consistent with the maximum nursing care score assigned to a specific care worker, which is determined according to the work capacity identified by the occupational physician;
f. not more than one new admitted patient;
g. patients who are hospitalised in neighbouring rooms;
h. patients who have similar isolation condition; and
i. patients who have isolation condition consistent with the health condition of the care workers.

15. The system according to claim 11, wherein the nursing care score of the assigned nursing work tasks has a value from 1.5 to 5.0.

16. The system according to claim 14, wherein the data processor is adapted to monitor the calculated total nursing care score to identify critical situations.

17. The system according to claim 9, wherein the data processor is adapted to interpret the assigned nursing work tasks for at least one patient to identify the required skill level, health and work capacity of the care workers and compare the required skill level, health and work capacity of the care workers with an existing available skill level, health and work capacity of the care workers.

18. The system according to claim 9, further comprising an alert generator for generating an alert message to a user in response to any identified critical situation.

19. A computer program product stored on a non-transitory computer readable medium and comprising:
a. a first logic for receiving health care requirement data of at least one patient;
b. a second logic for selecting at least one nursing work task with high nursing care related to the health care requirement data;
c. a third logic for assigning a nursing care score to the at least one selected nursing work task with high nursing care;
d. a fourth logic for calculating a total nursing care score from one or more of the assigned nursing care scores;
e. a fifth logic for receiving care workers' data; and
f. a sixth logic for assigning patients to care workers.

20. The computer program product according to claim 19, wherein the first logic is capable of adjusting the nursing care score for work tasks in the case a patient is isolated.
